(19) United States
(12) Patent Application Publication
(10) Pub. No.: US 2003/0195485 A1
(43) Pub. Date: Oct. 16, 2003

(54) Absorbent cores with improved intake performance

(75) Inventors: Krishnakumar Rangachari, Savannah, GA (US); Paul M. Ducker, St. Simons, GA (US)

Correspondence Address: ALSTON & BIRD LLP, BANK OF AMERICA PLAZA, 101 SOUTH TRYON STREET, SUITE 4000, CHARLOTTE, NC, 28280-4000, US

(73) Assignee: Rayonier Products and Financial Services Company

(21) Appl. No.: 10/411,604

(22) Filed: Apr. 10, 2003

Related U.S. Application Data
(60) Provisional application No. 60/372,743, filed on Apr. 12, 2002.

(51) Int. Cl. A61F 13/15; A61F 13/20

(52) U.S. Cl. 604/374; 604/367; 604/385.01

(57) ABSTRACT

Multilayered absorbent cores are provided that include synthetic fiber to improve the liquid transport properties of the resulting absorbent articles. The synthetic fiber, which may be found in either the innermost and/or intermediate layers of the absorbent core, particularly improve the rewet performance of the absorbent article. The absorbent cores may be incorporated into a number of absorbent articles, including diapers, feminine hygiene products and incontinence pads.

### CROSS-REFERENCE TO RELATED APPLICATIONS

This application claims the benefit of U.S. Provisional Application No. 60/372,743, filed Apr. 12, 2002 under 35 U.S.C. § 119(e), which is hereby incorporated herein in its entirety by reference.

### FIELD OF THE INVENTION

The present invention relates to absorbent materials for use in absorbent articles such as diapers and to processes by which to produce such absorbent materials. More particularly, the present invention relates to absorbent materials exhibiting improved liquid transport performance that further include synthetic fibers.

### BACKGROUND OF THE INVENTION

Absorbent articles are widely used in a variety of applications. To function efficiently, such absorbent articles must quickly absorb body fluids, distribute those fluids within and throughout the absorbent article and be capable of retaining those body fluids. In addition, the absorbent article should be sufficiently soft and flexible so as to comfortably conform to body surfaces and provide close fit for lower leakage.

Exemplary absorbent articles available in the market today include diapers, feminine hygiene products, incontinence pads, and the like. Almost all absorbent articles include at least three elements: a topsheet, a backing sheet and an absorbent core disposed therebetween. The topsheet, also commonly referred to as a “facing layer,” is positioned closest to the wearer. The topsheet passes liquids through its thickness, serves as containment means for the absorbent core and feels soft against the wearer's skin. The backing sheet, also referred to as a “backing layer,” is positioned directly adjacent to the wearer's undergarments. The backing sheet likewise serves as a containment means for the absorbent core, and also provides a waterproof barrier between the absorbent core and the wearer's undergarments following a liquid insult.

The absorbent core, also referred to as an absorbent panel, is generally designed to absorb and retain body exudates entering the absorbent article through the topsheet. The absorbent core is generally formed from hydrophillic fibers. For example, absorbent cores may be formed from cellulosic fibers, such as cellulosic fiber derived from wood pulp and the like. Absorbent cores derived from wood pulp fiber are widely used and commonly referred to in the art as “fluff pulp”.

Unfortunately, liquid insults generally impinge the topsheet, and are subsequently transferred to the absorbent core, in relatively small, localized areas. Further, the total amount of liquid delivered to these small areas can be quite significant. Such high delivery rates are problematic because the acquisition rate of the absorbent core is generally lower than the delivery rate of the liquid insult. Thus the absorbent capacity of the absorbent core within the area of liquid entry can quickly become overwhelmed, causing the liquid to pool until it is able to diffuse into the absorbent core over time. In addition, as the absorbent core becomes saturated by successive liquid insults, the intake performance of conventional absorbent cores dramatically decreases, further exacerbating the problem. More specifically, the acquisition rate of conventional absorbent cores generally decreases significantly with each successive liquid insult.

Absorbent gelling particles may be incorporated into the absorbent core to improve its acquisition rate. Unfortunately, gelling particles swell as they absorb the insult. The swollen particles diminish the void volume of the absorbent core, reducing its ability to rapidly absorb subsequent insults.

Optional liquid transport layers may be included within absorbent articles to facilitate the lateral spreading of the fluid, and further to rapidly transfer and distribute the insult to the absorbent core. The liquid transport layer, also commonly referred to as a transitional layer, transfer layer, acquisition layer or surge management layer, is typically disposed between the topsheet and absorbent core to help prevent the liquid from pooling and collecting on the portion of the absorbent article positioned against the wearer's skin, thus increasing the chance for leakage. Such liquid transport layers are generally porous, water permeable fabrics, formed from synthetic fibers. The liquid transport layers may be formed from synthetic fibers alone, or a blend of synthetic and natural fiber, e.g. cellulosic fiber. Exemplary liquid transport layers include nonwovens, such as meltblown webs, spunbonded webs, and the like. Such nonwovens generally have a low density (0.03 to 0.1 g/cc) or high loft. Although a separate liquid transport layer can generally satisfactory perform the above-described functions, the incorporation of a separate acquisition layer in an absorbent article complicates the structure and requires additional manufacturing steps. This also necessarily increases the cost of the final product.

Accordingly, there remains a need in the art for more economically produced absorbent articles having improved absorptive capabilities. More specifically, there remains a need in the art for absorbent articles which include absorbent cores possessing increased acquisition rates. There is also a need in the art for absorbent cores providing intake performances that either decrease less dramatically upon saturation and repeated insults in comparison to conventional absorbent cores or, advantageously, increase with successive liquid insults.

### BRIEF SUMMARY OF THE INVENTION

The present invention is directed to absorbent cores providing improved liquid transport performance, particularly increased acquisition rates, thus potentially eliminating the need for separate liquid transport layers. More specifically, Applicants have determined that the liquid transport properties of multi-layered absorbent cores may be improved, particularly over multiple insults, by including synthetic and/or regenerated staple fibers within one or more of the absorbent core layers, as indicated by increased acquisition rates and insult ratios in comparison to comparable absorbent cores without synthetic fiber. The synthetic and/or regenerated staple fibers can be incorporated into the absorbent core in the form of individualized fibers which are deposited as or within a layer during the absorbent core formation process, or the synthetic and/or regenerated staple fibers can be incorporated into the absorbent core in the form of a pre-formed nonwoven sheet.

The absorption performance of absorbent materials over time is commonly referred to as the “insult ratio”. The insult ratio as used herein refers to the acquisition rate after two or more insults divided by the initial acquisition rate. As further used herein, the term “second insult ratio” refers to the acquisition rate for the second insult divided by the initial acquisition rate. Similarly, as used herein the term “third insult ratio” refers to the acquisition rate for the third insult divided by the initial acquisition rate.

Applicants have determined that the beneficial acquisition rates of the present invention do not decrease as dramatically upon saturation and repeated insults as do conventional absorbent cores. Applicants have determined that the present invention generally provides second and third insult ratios of about 0.80 or higher. In fact, embodiments of the invention exhibit increased acquisition rates following saturation of the absorbent core and repeated liquid insults, i.e. second and third insult ratios greater than 1.0. Second and third insult ratios greater than 1.0 are altogether unexpected and heretofore unknown.

The invention generally provides absorbent cores that include (a) an innermost layer positioned towards the wearer that includes synthetic fiber in an amount effective to improve the liquid transport properties of said absorbent core; (b) at least one intermediate layer contiguous with the innermost layer and positioned away from the wearer, at least one of the intermediate layers including a mixture of cellulosic fiber and superabsorbent particles; and (c) an outermost layer containing cellulosic fiber that is contiguous with the intermediate layer and positioned furtherest from the wearer.

In alternative beneficial embodiments, the invention provides absorbent cores in which synthetic fiber is included within layers other than the innermost layer. For example, absorbent cores are provided that include (a) an innermost layer formed from cellulosic fiber positioned towards the wearer; (b) at least one intermediate layer contiguous with said innermost layer and positioned away from the wearer, at least one of the intermediate layers including synthetic fiber in an amount effective to improve the liquid transport properties of said absorbent core upon repeated liquid insults; and (c) an outermost layer formed from cellulosic fiber contiguous with the intermediate layer and positioned furtherest from the wearer.

The present invention further encompasses the methods by which to form absorbent cores including synthetic fiber and absorbent articles formed therefrom.

### BRIEF DESCRIPTION OF THE SEVERAL VIEWS OF THE DRAWING(S)

Having thus described the invention in general terms, reference will now be made to the accompanying drawings, which are not necessarily drawn to scale, and wherein:

FIG. 1 is a greatly enlarged, cross-sectional schematic view of one advantageous embodiment of the absorbent core of the present invention;

FIG. 2 is a greatly enlarged, cross-sectional schematic view of a second advantageous embodiment of the absorbent core of the present invention;

FIG. 3 is a simplified, diagrammatic view of an apparatus illustrating one advantageous process for making the improved absorbent core of the present invention;

FIG. 4 graphically illustrates the acquisition rate performance of conventional absorbent articles;

FIG. 5 graphically illustrates the acquisition rate performance of absorbent cores formed in accordance with beneficial embodiments of the present invention; and

FIG. 6 graphically illustrates the method by which the acquisition rate performance of the absorbent cores were determined.

### DETAILED DESCRIPTION OF THE INVENTION

The present inventions now will be described more fully hereinafter with reference to the accompanying drawings, in which some, but not all embodiments of the invention are shown. Indeed, these inventions may be embodied in many different forms and should not be construed as limited to the embodiments set forth herein; rather, these embodiments are provided so that this disclosure will satisfy applicable legal requirements. Like numbers refer to like elements throughout.

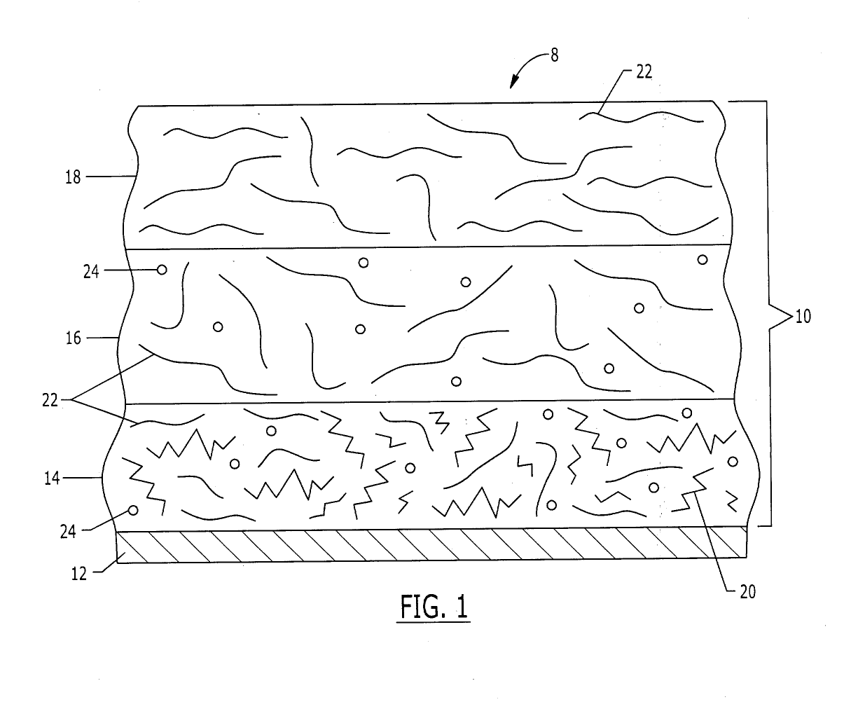

As illustrated in FIG. 1, the absorbent cores 8 of the present invention generally include a primary absorbent portion 10, disposed upon an optional carrier layer 12. The primary absorbent portion 10 typically includes at least three layers: an innermost layer 14, positioned closest to the wearer (and the carrier layer 12); one or more intermediate layers 16 (a single intermediate layer is illustrated in FIG. 1); and an optional outermost layer 18.

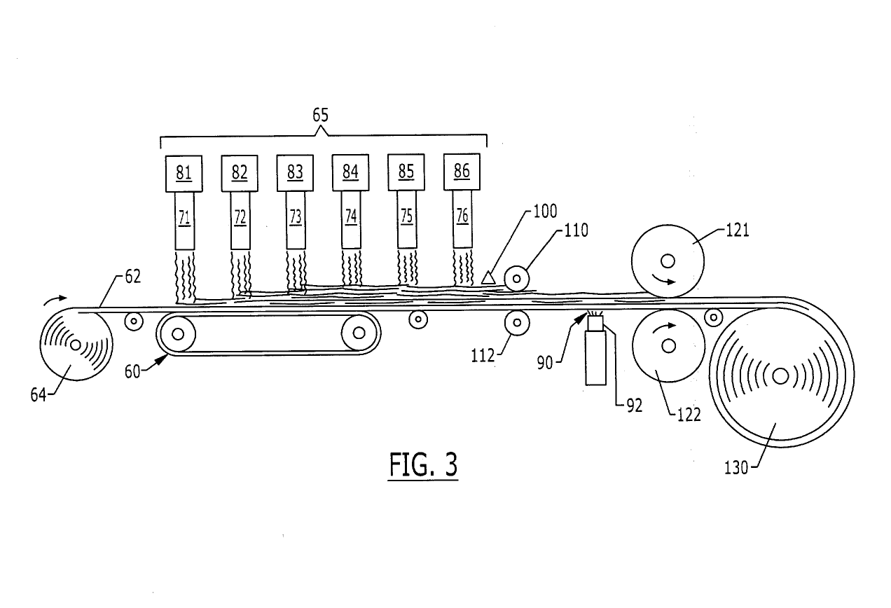

For the sake of clarity, the “layer count” will refer to the number of layers in the primary absorbent portion 10, i.e., the carrier layer 12 will not be included. For example, in the “three” layered embodiment of the invention provided in FIG. 3, the “three” layers are present within the primary absorbent portion 10, along with the carrier layer 12. Further, although the absorbent core is referred to as containing “layers,” this term is merely used to facilitate discussion concerning the differing compositions which may be present in various regions within the absorbent core thickness. The absorbent cores of the present invention, although referred to as being formed from such “layers,” nevertheless provide unitary structures exhibiting cohesive properties throughout their thickness. Further, each “layer” is generally in either direct or indirect liquid communication with its adjacent layer(s).

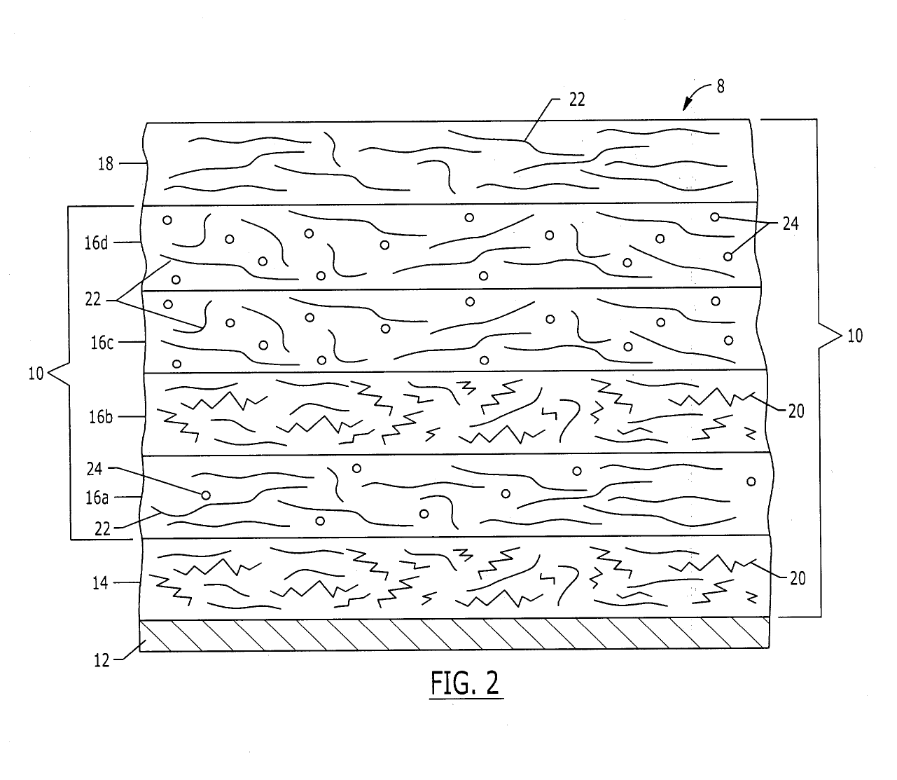

The innermost layer 14 of the absorbent core 8 typically includes synthetic and/or regenerated fibers 20, either alone or in combination with cellulosic fibers 22 and/or superabsorbent particles “SAP” 24, as illustrated in FIG. 1. The intermediate layers 16 are normally formed from a mixture of cellulosic fibers 22 and SAP 24, as further illustrated in FIG. 1. However, in aspects of the invention including multiple intermediate layers (as shown in FIG. 2) one or more of the intermediate layers 16 may also be formed from synthetic and/or regenerated fibers 20, either alone or in combination with cellulosic fibers 22 and/or SAP 24. In aspects of the invention in which one or more of the intermediate layers 16 includes synthetic and/or regenerated fibers 20, the innermost layer 14 may optionally be formed entirely from cellulosic fibers 22, either alone or in combination with SAP 24. As further shown in FIG. 1, the outermost layer 18 of the absorbent core 8 is typically formed entirely of cellulosic fiber 22.

Any known synthetic or regenerated fiber 20 known in the art may be incorporated into the absorbent cores 8 of the present invention, whether in the form individualized fibers or as a pre-formed nonwoven sheet. Advantageously, the synthetic fiber 24 is a thermoplastic fiber exhibiting a melting temperature of greater than about 170° C. Exemplary synthetic fibers include polyalkylene terephthalates, such as polyethylene terephthalate (“PET”); polyolefins, such as polyethylene (“PE”) and polypropylene (“PP”); acrylic; polyamides, such as nylon; and blends thereof. Exemplary regenerated fibers include rayon and acetate In advantageous embodiments, the synthetic fiber is polyethylene terephthalate. For the sake of brevity and clarity, the term “synthetic fiber” will be used hereinafter to refer to both synthetic and regenerated fibers.

The synthetic fibers of the invention may be included in the absorbent core in their natural state or may be hydrophillically modified. For example, the synthetic fibers may have either carboxyl or hydroxyl functionality grafted or coated onto its surface. The synthetic fiber may further have any known geometry. For example, the synthetic fiber may be either hollow or solid. The synthetic fiber may further have any cross-section known in the art of fiber formation. For example, the synthetic fiber may have a cross-section known to impart greater stiffness in comparison to circular fiber, such as quadralobal cross-sections or the like.

The synthetic fibers typically have a denier ranging from about 3 to 25 dpf, such as a denier of 3, 6, 9 or 15 dpf. (The term “dpf” refers to the weight in grams of 9,000 meters of a fiber.) The synthetic fibers are typically staple fibers. The synthetic fiber generally has a staple length of greater than about 2 mm, such as a nominal staple length ranging from about 2 to about 20 mm. In advantageous embodiments, synthetic fibers having a nominal staple length of about 6 mm are employed. As known in the art, staple fibers are typically crimped. In the instant invention, the synthetic fiber may be highly crimped. For example, the synthetic fibers may possess about 1 to 20 crimps/inch or greater.

The synthetic fibers may be present within the primary absorbent portion 10 in amounts ranging from about 10 to 100 gsm. For example, the synthetic fiber may be present in an absorbent core 8 having a basis weight of about 450 gsm in amounts ranging from about 10 to 100 gsm. In one advantageous embodiment, the synthetic fiber is present within an absorbent core having a basis weight of about 450 gsm in an amount of about 60 gsm. In further advantageous embodiments the synthetic fiber may be present in within absorbent cores having a basis weight of about 250 gsm in amounts ranging from about 10 to 60 gsm, such as a 250 gsm absorbent core containing 40 gsm synthetic fiber.

Considered on a relative weight basis, the synthetic fiber may thus beneficially be present with the absorbent core 8 in amounts ranging from about 2 to 30 weight percent, based on the weight of the absorbent core. (As used herein, the term “based on the weight of the absorbent core” may be abbreviated as “boc”). For example, the synthetic fiber may be present in the absorbent core in amounts ranging from about 13 to 16 weight percent, boc.

The total amount of synthetic fiber 20 may advantageously be present within the innermost layer 14, as shown in FIG. 1. In further beneficial embodiments, the synthetic fiber 20 may be portioned amongst the innermost layer and one or more intermediate layers 16. For example, one half of the total amount of the synthetic fiber 20 may be in the innermost layer 14 and the remaining half may be portioned amongst one or more intermediate layers 16. In alternative advantageous embodiments, the total amount of synthetic fiber may be present in the intermediate layer or a combination of the intermediate and outer layers, as well. Surprisingly, alternative embodiments in which synthetic fiber is present within the intermediate or intermediate and outer layers but not within the innermost layer similarly provide beneficial intake performances after repeated insults.

In advantageous embodiments, the synthetic-fiber is PET. For example, one or more layers within the primary absorbent portion 10 may include PET fibers having a nominal 6 millimeter staple length and about 15 dpf in a highly crimped condition. Absorbent materials made in accordance with the present invention may also include PET fibers having a nominal staple length of 6 millimeters and 9 dpf in a highly crimped condition, as well as PET fibers having nominal length of 6 millimeters and 3 dpf in a highly crimped condition. In beneficial aspects of these embodiments, PET fiber is included within the innermost layer 14 of the primary absorbent portion 10. In further advantageous embodiments, PET fiber is included within the innermost layer 14 and at least one intermediate layer 16. In alternative embodiments, PET fiber is included within either (a) at least one intermediate layer 16 and the outermost layer 18 or (b) at least one intermediate layer 16, but not within the innermost layer 14. The PET fiber could have any known geometry, for example, the PET fiber could be either a hollow fiber or a solid fiber.

The present invention also contemplates the use of multicomponent synthetic fibers in one or more layers of the primary absorbent portion 10. Exemplary multicomponent fibers include bicomponent fibers, such as bicomponent PP/PE fiber or PP/PET fibers. One example of PP/PE bicomponent fiber suitable for use in the present invention includes a polypropylene core and a polyethylene sheath and has a nominal staple length of 6 millimeters and 10 to 12 denier. An exemplary PP/PET fiber includes a PET core and PP sheath with a nominal staple length of about 6 mm and 12 dpf.

The synthetic fibers described above can be incorporated into the absorbent core in the form of individualized fibers which are deposited so as to form at least a portion of a layer during the absorbent core formation process. In alternative advantageous embodiments, the synthetic staple fibers described above can be incorporated into the absorbent core in the form of a pre-formed nonwoven sheet or web. As used herein, the term “sheet” is used interchangeably with the term “web.” Any nonwoven construction known in the art may be used as the pre-formed web. Suitable pre-formed nonwoven webs are typically formed from fiber having a denier ranging from about 3 to 25 dpf and fiber lengths ranging from about 2 to 20 mm. Pre-formed nonwoven sheets suitable for use in the invention also generally exhibit a basis weight ranging from about 20 to 80 gsm. Any of the bonding technologies well known in the art, including but not limited to through-air-bonding (“TAB”), spunbonding, chemical bonding, thermal point bonding, needle punching and hydroentanglement, may be used to form the pre-formed nonwoven web. One exemplary suitable material is a TAB nonwoven sheet commercially available as Dry-web T-9, a 40 gsm basis weight web available from Libeltex N. V. of Meulebeke, Belgium. The pre-formed nonwoven sheets may generally form the innermost layer and/or one or more of the intermediate layers. The pre-formed sheet may generally form from about 4 to 32 weight percent of the absorbent core, such as from about 8 to 16 weight percent of the absorbent core.

Cellulosic fibers 22 are included in at least the outermost layer 18 and one or more of the intermediate layers 16. Cellulosic fibers 22 may optionally be included in the innermost layer 14, as well. Cellulosic fibers that can be used in the absorbent articles of the present invention are well known in the art and include fiber derived from wood pulp, cotton, flax, and peat moss. In advantageous embodiments, cellulosic fiber derived from wood pulp is employed. Wood pulp fibers can be obtained from mechanical or chemi-mechanical, sulfite, kraft, pulping reject materials, organic solvent pulps, etc. Both softwood and hardwood species are useful. Softwood pulps are preferred. It is not generally necessary to treat cellulosic fibers with chemical debonding agents, cross-linking agents and the like for use in the primary absorbent portion, although such treatments may be employed.

Advantageously, the wood pulp is prepared using a process that reduces the lignin content of the wood. For example, the lignin content of the pulp may be less than about 16 percent, such as a lignin content of less than about 10 percent. Beneficially, the lignin content is less than about 5 percent, such as a lignin content of less than about 1 percent. As is well known in the art, lignin content is calculated from the Kappa value of the pulp. The Kappa value is determined using a standard, well known test procedure TAPPI Test 265-cm 85. The Kappa value of a variety of pulps was measured and the lignin content calculated using the TAPPI Test 265-cm 85. The cellulosic fibers of the present invention may advantageously be derived from wood pulp having a Kappa value of less than about 100. Beneficially, the Kappa value is less than about 75, such as a Kappa value of less than 50 and beneficially less than 25, 10 or 2.5.

In one advantageous embodiment, the cellulosic fiber is derived solely from standard untreated cellulose. In further beneficial embodiments, the cellulosic fiber may be a mixture of standard untreated cellulosic fibers and alkaline treated cellulosic fibers, such as cold caustic treated (“CCT”) cellulosic fibers. The weight ratio of standard untreated cellulosic fiber to alkaline treated cellulosic fiber may beneficially range from about 0:100 to 100:0, such as 0.5:1 to 10:1. For example, in advantageous embodiments the weight ratio of standard untreated cellulosic fiber to alkaline treated cellulosic fiber may range from about 1.2:1 to 1.29:1. Considered differently, a mixture of standard untreated cellulosic fibers and alkaline treated cellulosic fibers may be employed in which the untreated cellulosic fibers are present in an amount ranging from about 15 to 30 weight percent, bol, such as from about 19 to 27 weight percent, bol, while the alkaline treated cellulosic fibers may be present in amounts ranging from about 15 to 25 weight percent, bol, such as from about 17 to 22 weight percent, bol.

Alkaline treatments for cellulosic fiber, particularly wood pulp fibers, are well known in the art. By way of example, treating wood pulp with liquid ammonia is known to decrease relative crystallinity and to increase the fiber curl value. Alternatively, cold caustic treatment of wood pulp also increases fiber curl and decreases relative crystallinity.

A description of absorbent cores containing cold caustic treated cellulosic fibers is described in commonly owned U.S. Pat. Nos. 5,866,242 and 5,916,670, both of which are incorporated in their entirety herein by reference thereto. Cold caustic treated cellulosic fibers are commercially available. Exemplary commercially available cold caustic treated cellulosic fiber is POROSANIER-BAT™ fiber from Rayonier, Inc. of Jesup, Ga.

Briefly, in cold caustic treatment a caustic treatment is typically carried out at a temperature less than about 60° C., advantageously at a temperature less than 50° C., such as a temperature between about 10° C. and about 40° C. One exemplary alkali metal salt solution is a sodium hydroxide solution newly made up or as a solution by-product in a pulp or paper mill operation, e.g., hermicaustic white liquor, oxidized white liquor and the like. Other alkali metals such as ammonium hydroxide and potassium hydroxide and the like can be employed. However, from a cost standpoint, sodium hydroxide may advantageously be utilized. The concentration of alkali metal salts is typically in a range from about 2 to about 25 weight percent of the solution, and preferably from about 6 to about 18 weight percent. Pulps for high rate, fast absorbing applications are generally treated with alkali metal salt concentrations from about 10 to about 18 weight percent. In alternative embodiments, methods other than alkaline treatment may be used to produce wood pulp fiber exhibiting lower crystallinity and increased curl. For example, flash dried or chemically cross-linked wood pulp may be employed.

As noted above, cellulosic fiber 22 may generally be present in several of the layers within the primary absorbent portion 10, including the outermost layer 18, one or more intermediate layers 16 and, optionally, the innermost layer 14. The outermost layer 18 may contain cellulosic fiber in amounts ranging from about 20 to 100 wt %, based on the weight of the layer. (As used herein, the term “based on the weight of the layer” may be abbreviated “bol”.) In beneficial embodiments, the outermost layer 18 may be formed entirely of cellulosic fiber. Cellulosic fiber 22 may be present within one or more of the intermediate layers 16 in amounts ranging from about 0 to 100 weight percent, bol, such as in amounts ranging from about 20 to 100 weight percent, bol. In embodiments including more than one intermediate layer 16, the cellulosic fiber 22 may be equally portioned amongst the layers. Alternatively, the cellulosic fiber may be present in greater amounts in intermediate layers positioned closest to the wearer. Cellulosic fiber 22 may also be present within the innermost layer 14, in amounts of up to about 50 weight percent, bol. In one beneficial embodiment, cellulosic fiber 22 is included in the innermost layer 14 in an amount of about 29 weight percent, bol. In the alternative embodiments of the invention in which one or more pre-formed nonwoven sheets is used to form one or more of the layers, the amount of cellulosic fiber within a given pre-formed sheet range from about zero to 90 weight percent, bol.

Superabsorbent particles (“SAP”) 24 may be included within one or more of the intermediate layers 16 and, optionally, the innermost layer 14. As used herein, the term “superabsorbent particle” includes any substantially water-insoluble polymeric material capable of absorbing large quantities of fluid in relation to its weight. The SAP can be in the form of particulate matter, flakes, fibers and the like. Exemplary particulate forms include granules, pulverized particles, spheres, aggregates and agglomerates. Exemplary SAP include polyacrylamides, polyvinyl alcohol, polyacrylates, various grafted starches, and the like. In advantageous embodiments, the superabsorbent materials include salts of crosslinked polyacrylic acid such as sodium polyacrylate. Superabsorbent materials are commercially available. Exemplary commercially available SAPs include SXM 880 and SXM 9200, both of which are available from Stockhausen GmbH, Krefeld, Germany.

The total amount of SAP present within the absorbent core may range from about 10 to 60 weight percent based on the weight of the absorbent core. For example, the SAP may be present in the absorbent core in an amount ranging from about 25 to 60 weight percent, such as in an amount of about 55 weight percent. SAP may be beneficially incorporated into the innermost layer 14, in amounts ranging up to about 70 weight percent, bol, such as from about 25 to 65 weight percent, bol. In one advantageous embodiment, SAP may be included in the innermost layer 14 in an amount of about 29 weight percent, bol. SAP may be beneficially incorporated into the intermediate layer 16 in amounts ranging from about 0 to 85 weight percent, such as from about 5 to 67 weight percent, beneficially about 39 weight percent, bol.

The concentration of superabsorbent particles is generally uniform along the length of the instant absorbent cores. However, in beneficial embodiments various SAP concentration gradients may be employed through the thickness of the absorbent core. For example, in embodiments directed to multiple intermediate layers, the total amount of SAP is generally portioned amongst two or more intermediate layers. For example, the SAP may be divided equally amongst several intermediate layers. Alternatively, the SAP may be present in lesser amounts in intermediate layers positioned closest to the wearer. In further alternative embodiments, the total amount of SAP may be distributed amongst several intermediate layers in a parabolic fashion.

A number of exemplary materials may be employed as the carrier layer. The carrier layer 12 may be, for example, either a spunbond or melt-blown non-woven consisting of natural or synthetic fibers.

Tissue may also be advantageously used as the carrier layer 12. Suitable tissue materials for use as a carrier layer 12 in absorbent cores 8 are well known to those of ordinary skill in the art. Beneficially, such tissue is made of bleached wood pulp and has an air permeability of about 273-300 CFM (cubic feet minute). The tensile strength of the tissue may be such that it retains integrity during formation and other processing of the absorbent material. Suitable MD (machine direction) and CD (cross direction) tensile strengths, expressed in newtons/meter, are about 100-130 and 40-60, respectively. The tissue may be a crepe tissue having a sufficient number of crepes per inch to allow a machine direction elongation of between 20 and 35 percent (as determined by the SCAN P44:81 test method). The basis weight of the carrier layer 22 is typically between about 15 and about 20 g/m², but could be more or less. Tissue for use in air-laying absorbent materials are commercially available (e.g., from Cellu Tissue Corporation, 2 Forbes Street, East Hartford, Conn. 06108, U.S.A., and from Duni A B, Sweden). In an alternative embodiment, a top carrier layer (not shown in FIG. 1) may further be disposed on the outermost layer 18. Such a top carrier layer may be formed from the same or different material than the bottom carrier layer 12.

The innermost layer 14 may compose about 3 to 20 weight percent of the absorbent core. For example, the innermost layer 14 may constitute about 7 to 16 weight percent of the absorbent core. The intermediate layer 16 may compose about 20 to 90 weight percent of the absorbent core. For example, the intermediate layer 16 may constitute about 69 to 92 weight percent of the absorbent core. The outermost layer 18 may compose about 0 to 20 weight percent of the absorbent core, such as from about 2 to 15 weight percent of the absorbent core. For example, the outermost layer 18 may constitute about 4 weight percent of the absorbent core. The carrier layer 22 may compose from about 1 to 10 weight percent of the absorbent core, such as from about 3 to 8 weight percent of the absorbent core.

FIG. 2 illustrates a beneficial embodiment in which the absorbent core 8 is formed from six (6) layers. In such six layer constructions, the innermost layer 14 may generally comprise from about 5 to 33 weight percent of the absorbent core. In advantageous aspects of these embodiments, the innermost layer 14 may comprise between 7 to 16 weight percent of the absorbent core, particularly about 7 weight percent of the absorbent core.

As shown in FIG. 2, the innermost layer 14 typically includes synthetic fiber 20. The synthetic fiber 20 may advantageously be present within the innermost layer 14 in amounts ranging from about 20 to 80 gsm, for absorbent cores ranging in basis weight from 250 to 450 gsm. On a relative weight basis, the synthetic fiber 20 may generally be present within the innermost layer 14 in amounts ranging from about 20 to 100 weight percent bol, such as in amounts ranging from about 43 to 100 weight percent bol, particularly in an amount of about 100 weight percent bol.

Advantageously, the innermost layer 14 may be formed from a combination of synthetic fiber, cellulosic fiber and optional SAP (not shown in FIG. 2). In such advantageous embodiments, the cellulosic fiber 22 and SAP 24 may each independently be included in the innermost layer 14 in amounts of up to about 50 weight percent bol, such as an amount of about 29 weight percent bol.

The construction illustrated in FIG. 2 includes a plurality of intermediate layers 16, designated 16a through 16d. Layers 16a, 16c and 16d are typically formed from a mixture of cellulosic fiber and SAP.

The first intermediate layer 16a may constitute from about 0 to 50 weight percent of the absorbent core, such as from about 5 to 50 weight percent of the absorbent core. Advantageously, the first intermediate layer 16a comprises from about 0 to 26 weight percent of the absorbent core, such as about 14 weight percent of the absorbent core.

The first intermediate layer 16a may contain cellulosic fiber 22 in amounts ranging from about 15 to 100 weight percent bol, advantageously in an amount ranging from about 33 to 100 weight percent bol. In advantageous embodiments, the first intermediate layer 16a includes cellulosic fiber 22 in an amount of about 61 weight percent, bol. The first intermediate layer 16a may further contain SAP 24 in amounts ranging from about 0 to 85 weight percent bol, such as in amounts ranging from 5 to 67 weight percent bol. In beneficial embodiments, the first intermediate layer 16a includes SAP 24 in an amount of about 39 weight percent bol. The first intermediate layer 16a may also contain synthetic fiber in amounts of up to 50 weight percent, bol, such as about 43 weight percent, bol.

The third and fourth intermediate layers 16c and 16d may each independently comprise from about 12 to 70 weight percent of the absorbent core. Advantageously, the third and fourth intermediate layers 16c and 16d may each independently comprise from about 24 to 35 weight percent of the absorbent core. In beneficial embodiments, intermediate layer 16c may comprise 32 weight percent of the absorbent core and intermediate layer 16d may comprise 33 weight percent of the absorbent core.

The third and fourth intermediate layers 16c and 16d generally contain cellulosic fiber 22 in amounts ranging independently from about 10 to 66 weight percent bol, such as an amount ranging from about 20 to 33 weight percent bol. In advantageous embodiments, the third intermediate layer 16c includes cellulosic fiber in an amount of about 23 weight percent bol and the fourth intermediate layer 16d includes cellulosic fiber in an amount of about 22 weight percent bol.

The third and fourth intermediate layers 16c and 16d may further contain SAP 24 in amounts ranging independently from about 33 to about 90 weight percent bol, such as amounts ranging from about 67 to 80 weight percent bol. In beneficial embodiments, the third intermediate layer 16c includes SAP in an amount of about 77 weight percent bol and fourth intermediate layer 16d includes SAP in an amount of about 78 weight percent bol.

The third and fourth intermediate layers 16c and 16d may further independently contain synthetic fiber in amounts ranging from about 0 to 100 weight percent, bol, such as from about 5 to 100 weight percent, bol. In advantageous embodiments, the third and fourth intermediate layers 16c and 16d may independently contain from about 30 to 40 weight percent synthetic fiber, bol, such as from about 33 to 38 weight percent synthetic fiber, bol.

The second intermediate layer 16b, which is an optional layer, may be formed from synthetic fiber 20, either alone or in combination with cellulosic fiber 22 and/or SAP 24. In alternative beneficial embodiments, the second intermediate layer 16b may be formed from cellulosic fiber 22, alone or in combination with SAP 24, i.e. without the inclusion of synthetic fiber 20.

The second intermediate layer 16b may comprise from about 0 to 33 weight percent of the absorbent core. Advantageously, the second intermediate layer 16b may to comprise from about 0 to 16 weight percent of the absorbent core. In one beneficial embodiment, the second intermediate layer 16b may comprise 7 weight percent of the absorbent core.

The second intermediate layer 16b may contain synthetic fiber 20 in amounts ranging from about 0 to 100 weight percent bol. For example, the second intermediate layer 16b may contain synthetic fiber 20 in an amount of about 20 to 100 weight percent bol, such as an amount of about 100 weight percent, bol.

The second intermediate layer 16b may further include cellulosic fiber 22 and/or SAP 24 in amounts ranging from about 0 to 60 weight percent bol, such amounts ranging from 0 to 29 weight percent, bol.

The outermost layer 18 may generally comprise from about 0 to 10 weight percent of the absorbent core. In advantageous aspects of these embodiments, the outermost layer 14 may comprise about 4 weight percent of the absorbent core. The outermost layer 18 may advantageously contain from about 20 to 100 weight percent bol of cellulosic fiber 22. In beneficial embodiments, the outermost layer 18 includes about 100 weight percent cellulosic fiber 22.

The absorbent core 8 generally exhibits a basis weight ranging from about 100 to 800 gsm. As known in the art, higher basis weight constructions, such as 450 gsm constructions, are generally well suited for diaper applications. Lower basis weight constructions, such as 250 gsm constructions, may be preferable for adult incontinence and feminine care applications.

The moisture content of the absorbent core 8 after equilibration with the ambient atmosphere is generally less than about 10% (by weight of the total material weight), such as less than about 8%, and beneficially lies in the range of between about 1% and 8%. A typical thickness of the absorbent core 8 is between 0.5 mm and 2.5 mm.

The density of the absorbent core 8 is generally greater than or equal to about 0.18 g/cm³. The density of the absorbent core 8 advantageously ranges from between about 0.2 and 0.5 g/cm³ such as from about 0.25 to 0.40 g/cm ³. The density of conventional absorbent cores is typically much lower than the present absorbent cores. For example, U.S. Pat. No. 5,913,850 to D'Alessio et al. notes the use of absorbent cores having a bulkiness of 20 cc/g, translating to a density of 0.05 g/cm³. Such lower density conventional cores would be expected to provide greater void volume and hence better liquid transport properties. It is thus altogether surprising that the instant absorbent cores, generally exhibiting higher densities than conventional absorbent cores, would provide advantageous liquid transport properties in comparison to conventional cores, particularly improved second and/or third insult ratios.

Surprisingly, by carefully tailoring the components within the various layers of the absorbent core, Applicants have produced absorbent cores exhibiting second, and even third, insult ratios of greater than about 0.8, and advantageously greater than about 0.90. In contrast, conventional absorbent cores typically provide insult ratios of less than 0.60. Applicants have further found that absorbent cores formed in accordance with the invention can exhibit second insult ratios of greater than about 1.0, such as ratios of greater than about 1.2 or 1.5. The beneficial absorption properties of the invention are provided for the third insult ratio, as well. More specifically, absorbent cores formed in accordance with the invention can similarly exhibit third insult ratios of greater than 1.0, such as a ratio of 1.2 or more, or even 1.3 or more. Insult ratios of greater than 1.0 indicate that the acquisition rate of later insults was higher than the acquisition rate of the initial insult. Such behavior is altogether surprising and has heretofore been unknown. The absorbent cores of the invention also advantageously provide initial acquisition rates, also referred to as intake rates, of greater than about 0.70 ml/sec, such as initial acquisition rates of greater than 0.9 or 1.0 ml/sec.

The instant absorbent cores may be formed by any means known in the art. For example, the absorbent cores may be produced by manufacturing processes which employ forming wires, screens or belts, such as air laying or wet laying techniques. FIG. 3 schematically illustrates an advantageous air laying process by which to produce absorbent core in accordance with the invention. More specifically, FIG. 3 illustrates a process by which to air lay a six layer construction (such as the construction illustrated in FIG. 2). Air laying is commonly used in conjunction with wood pulp. To air lay a layer of wood pulp, incoming wood pulp is initially separated into individualized wood fibers, using a hammer mill or the like (not shown). In general, the individualized wood fibers are transported through a forming head station 65 and deposited by vacuum onto a forming wire 60.

The process permits the optional incorporation of a bottom carrier layer 62 in the absorbent material (e.g., carrier layer 12 in the absorbent material described above with reference to FIGS. 1 and 2, respectively). To this end, as shown in FIG. 3, a carrier web 62 is unwound from a carrier web roll 64 and directed over the endless forming wire 60. A series of forming heads in a forming head station 65 are provided over the endless forming wire 60. The illustrated forming head station 65 includes first through sixth forming heads 71 and 76. In alternative embodiments, a lesser or greater number of forming heads may be provided. For example, the station may include as few as 2 forming heads.

In advantageous embodiments, the first forming head 71 discharges synthetic fiber alone. Alternatively, the first forming head 71 may discharge a blend of synthetic fiber and cellulosic fiber, optionally containing SAP. In further alternative embodiments that include synthetic fiber within one or more of the intermediate layers, the first forming head 71 may discharge cellulosic fiber, either alone or in combination with SAP. The intermediate forming heads 72 through 75 typically discharge cellulosic fiber, beneficially in combination with SAP. In one beneficial embodiment, an intermediate forming head, such as forming head 73, discharges synthetic fiber in lieu of or in addition to cellulosic fiber and/or SAP. In an alternative beneficial embodiment, one or more of the intermediate forming heads, such as forming head 73, stands idle and does not deposit a layer of fiber upon the intermediate construction. Advantageously, the final forming head, illustrated as forming head 76 in FIG. 3, discharges only cellulosic fiber without discharging synthetic fibers or SAP.

The blending and distribution of the various components, i.e., the synthetic fiber, cellulosic fiber and SAP, can be controlled separately for each forming head. The forming head 71 is connected with a blending system 81, and the forming head 72 is connected with a blending system 82, and so on, through forming head 76, connected with a blending system 86. The pulp fibers, synthetic polymer fibers, and superabsorbent granules or particles can be blended in the blending systems and conveyed pneumatically into the appropriate forming heads. Alternatively, the pulp fibers, synthetic polymer fibers, and superabsorbent granules or particles can be conveyed separately to the appropriate forming heads and then blended together in the forming heads. Controlled air circulation and winged agitators in each blending system may be used to produce a substantially uniform mixture and distribution of the pulp and superabsorbent particles and/or synthetic polymer fibers.

The material from each forming head is deposited, preferably with vacuum assist, as a loose, uncompacted, layer superposed on the preceeding layer. The first layer, deposited by forming head 71, is advantageously deposited directly on the carrier layer 62 (or, alternatively, directly onto the endless screen 60). Although not wishing to be bound by theory, Applicants hypothesize that the carrier layer 62 provides a natural barrier to hold the synthetic fiber in position, thereby avoiding dust formation. Applicants further hypothesize that the outer layers of the absorbent core, e.g., the layers produced by forming heads 72 through 76, provide a similar function. Thus, the synthetic fiber deposited by the initial forming head 71 resides in a containment means defined by the carrier layer 62 and subsequent absorbent core layers issuing from forming heads 72-76.

In alternative advantageous embodiments of the invention (not shown), one or more pre-formed nonwoven sheets, generally in the form of roll goods, can be introduced between any of the forming heads 71 through 76 or between the carrier layer 12 and the first forming head 71. In such alternative advantageous embodiments employing preformed nonwoven sheet, the integrity of the pre-formed sheet prevents the synthetic fibers from dusting.

In advantageous embodiments, the carrier layer 62 may be subjected to an optional water spray 90 provided by nozzle 92. The water spray 90 is believed to promote bonding between the carrier layer 62 and the cellulosic fibers present within the absorbent core. In further beneficial aspects of this embodiment, SAP is included within the synthetic fiber deposited by the first forming head 71, to further enhance bonding between the carrier layer 62 and cellulosic fibers during product usage.

The loose layers of absorbent core are then conveyed, preferably with the help of a conventional vacuum transfer device 100, from the end of the endless screen 60 through a first set of compaction rolls 110 and 112 and then through calendar rolls. The calendar rolls include an upper roll 121 and a lower roll 122 which compress or compact the absorbent core to form an increased density web.

In one advantageous embodiment, the upper roll 121 is typically a steel roll, and the lower roll 122 is typically a steel roll. In beneficial aspects of the invention, the upper roll 121 has an embossing pattern surface, and the lower roll 122 has a smooth surface. In some applications it may be desirable to reverse the orientation of the web through the rolls so that the embossing roll contacts the carrier layer 62 of the web. In other applications, it may be desirable to provide both the upper and lower rolls 121 and 122 with an embossing pattern surface.

The weight of the upper roll 121 bears on the web. Additional force may be provided with conventional hydraulic actuators (not illustrated) acting on the axle of the roll 121. In one form of the invention, the web is compacted between the rolls 121 and 122 under a load of between about 28 and about 400 newtons per millimeter of transverse web width (160-2284 pounds force per inch of transverse web width).

The processing line is preferably run at a line speed of between about 30 meters per minute and about 300 meters per minute. Either one or both of rolls 121 and 122 may be heated. In advantageous aspects, each of rolls 121 and 122 is heated, in beneficial embodiments, to at least about 120° C. In one advantageous embodiment, the calendar rolls 121, 122 are heated to a temperature ranging from about 120 to 170° C. The temperature of the rolls 121 and 122 should be sufficient to facilitate the establishment of hydrogen bonding of the pulp fibers to each other, as well as of the tissue layer (if any) to the pulp fibers, so as to increase the strength and integrity of the finished absorbent core. The calendaring of the present invention provides a finished absorbent core with exceptional strength and resistance to shake-out of synthetic fiber and superabsorbent material.

The temperature of each roll is dependent upon the line speed and type of synthetic polymer fiber that is employed. It has been found that the process of the present invention can be operated to provide absorbent cores which, while having improved fluid acquisition properties imparted by the synthetic fibers, still has a relatively low Gurley Stiffness and is therefore soft and supple.

According to preferred forms of the invention, the temperatures of the rolls 121 and 122 are not sufficient to cause melting of the surface of the synthetic fibers incorporated in the web at the particular line speed and compaction load that are employed. By avoiding the melting of the surfaces of the synthetic polymer fibers, the process minimizes the formation of thermal bonds that would increase rigidity and stiffness of the web.

Upon leaving the rolls 121 and 122, the web contains very little moisture (e.g., 1%-8% moisture based on the total weight of the web). The compressed and densified web is wound into a roll 130 using conventional winding equipment. The web moisture content will typically increase as the web reaches equilibrium with the ambient atmosphere, but it is desirable that the moisture content not be too high--advantageously the web moisture content ranges between about 1% and about 8% of the total weight of the web.

The high density absorbent cores made by the process of the present invention, typically containing synthetic fibers within their innermost layer, have good fluid acquisition and absorptive capabilities, are surprisingly and unexpectedly soft and supple, and yet are relatively strong with good integrity, both wet and dry. The absorbent cores can be prepared by the process of the present invention over a wide range of basis weights without adversely affecting their softness or strength.

The invention will be further illustrated by the following non-limiting examples.

### EXAMPLES

Examples 1 through 9 in accordance with present invention and Comparative Examples 1 through 8 were produced using the layer compositions provided as Recipes A through J below. The specific recipes used for each of the Examples 1 through 9 and Comparative Examples 1 through 8 are noted in Table 1. The samples were produced using 17 gsm tissue as the carrier layer, commercially available as designated grade 3008 from Cellu Tissue Corporation. The SAP, both the SXM 880 and the SXM 9200 were obtained from Stockhansen GmbH, Krefeld, Germany. The PET was hydrophillically treated fiber having a nominal staple length of 6 mm and denier and geometries described in Table 11. The PET was procured from KOSA of Charlotte, N.C. The cellulose fiber was untreated pulp fiber identified as RAYFLOC-J-LD pulp fiber, commercially available from Rayonier Inc. of Jesup, Ga.

The samples were prepared using the process described in conjunction with FIG. 3, with FH1 through FH6 corresponding to forming heads 71 through 76, respectively. Water was applied to the carrier sheet prior to calendaring in an amount of about 1 weight percent boc for samples having a basis weight about 250 gsm and in an amount of about 7 weight percent boc for all other samples.

| % in Each Forming Head | | | % of total |
|---|---|---|---|
| SAP | PET | Pulp | basis weight |

RECIPE A

| | | | | |
|---|---|---|---|---|
| Tissue | | | | 4% |
| FH 1 | 0% | 0% | 100% | 13% |
| FH 2 | 67% | 0% | 33% | 26% |
| FH 3 | 0% | 0% | 0% | 0% |
| FH 4 | 73% | 0% | 27% | 26% |
| FH 5 | 73% | 0% | 27% | 26% |
| FH 6 | 0% | 0% | 100% | 4% |

RECIPE B

| | | | | |
|---|---|---|---|---|
| Tissue | | | | 4% |
| FH 1 | 0% | 100% | 0% | 13% |
| FH 2 | 67% | 0% | 33% | 26% |
| FH 3 | 0% | 0% | 0% | 0% |
| FH 4 | 73% | 0% | 27% | 26% |
| FH 5 | 73% | 0% | 27% | 26% |
| FH 6 | 0% | 0% | 100% | 4% |

RECIPE C

| | | | | |
|---|---|---|---|---|
| Tissue | | | | 4% |
| FH 1 | 0% | 0% | 100% | 13% |
| FH 2 | 0% | 0% | 100% | 9% |
| FH 3 | 0% | 0% | 0% | 0% |
| FH 4 | 80% | 0% | 20% | 35% |
| FH 5 | 80% | 0% | 20% | 35% |
| FH 6 | 0% | 0% | 100% | 4% |

RECIPE D

| | | | | |
|---|---|---|---|---|
| Tissue | | | | 4% |
| FH 1 | 0% | 100% | 0% | 13% |
| FH 2 | 0% | 0% | 100% | 9% |
| FH 3 | 0% | 0% | 0% | 0% |
| FH 4 | 80% | 0% | 20% | 35% |
| FH 5 | 80% | 0% | 20% | 35% |
| FH 6 | 0% | 0% | 100% | 4% |

RECIPE E

| | | | | |
|---|---|---|---|---|
| Tissue | | | | 7% |
| FH 1 | 0% | 0% | 100% | 16% |
| FH 2 | 38% | 0% | 62% | 21% |
| FH 3 | 0% | 0% | 0% | 0% |
| FH 4 | 67% | 0% | 33% | 24% |
| FH 5 | 67% | 0% | 33% | 24% |
| FH 6 | 0% | 0% | 100% | 8% |

RECIPE F

| | | | | |
|---|---|---|---|---|
| Tissue | | | | 7% |
| FH 1 | 0% | 100% | 0% | 16% |
| FH 2 | 38% | 0% | 62% | 21% |
| FH 3 | 0% | 0% | 0% | 0% |
| FH 4 | 67% | 0% | 33% | 24% |
| FH 5 | 67% | 0% | 33% | 24% |
| FH 6 | 0% | 0% | 100% | 8% |

RECIPE G

| | | | | |
|---|---|---|---|---|
| Tissue | | | | 4% |
| FH 1 | 0% | 100% | 0% | 7% |
| FH 2 | 39% | 0% | 61% | 14% |
| FH 3 | 0% | 100% | 0% | 7% |
| FH 4 | 77% | 0% | 23% | 32% |
| FH 5 | 78% | 0% | 22% | 33% |
| FH 6 | 0% | 0% | 100% | 4% |

RECIPE H

| | | | | |
|---|---|---|---|---|
| Tissue | | | | 4% |
| FH 1 | 0% | 0% | 100% | 7% |
| FH 2 | 39% | 0% | 61% | 14% |
| FH 3 | 0% | 0% | 100% | 7% |
| FH 4 | 77% | 0% | 23% | 32% |
| FH 5 | 78% | 0% | 22% | 33% |
| FH 6 | 0% | 0% | 100% | 4% |

RECIPE I

| | | | | |
|---|---|---|---|---|
| Tissue | | | | 4% |
| FH 1 | 29% | 43% | 29% | 16% |
| FH 2 | 0% | 0% | 0% | 0% |
| FH 3 | 29% | 43% | 29% | 16% |
| FH 4 | 77% | 0% | 23% | 30% |
| FH 5 | 77% | 0% | 23% | 30% |
| FH 6 | 0% | 0% | 100% | 4% |

RECIPE J

| | | | | |
|---|---|---|---|---|
| Tissue | | | | 4% |
| FH 1 | 29% | 0% | 71% | 16% |
| FH 2 | 0% | 0% | 0% | 0% |
| FH 3 | 29% | 0% | 71% | 16% |
| FH 4 | 77% | 0% | 23% | 30% |
| FH 5 | 77% | 0% | 23% | 30% |
| FH 6 | 0% | 0% | 100% | 4% |

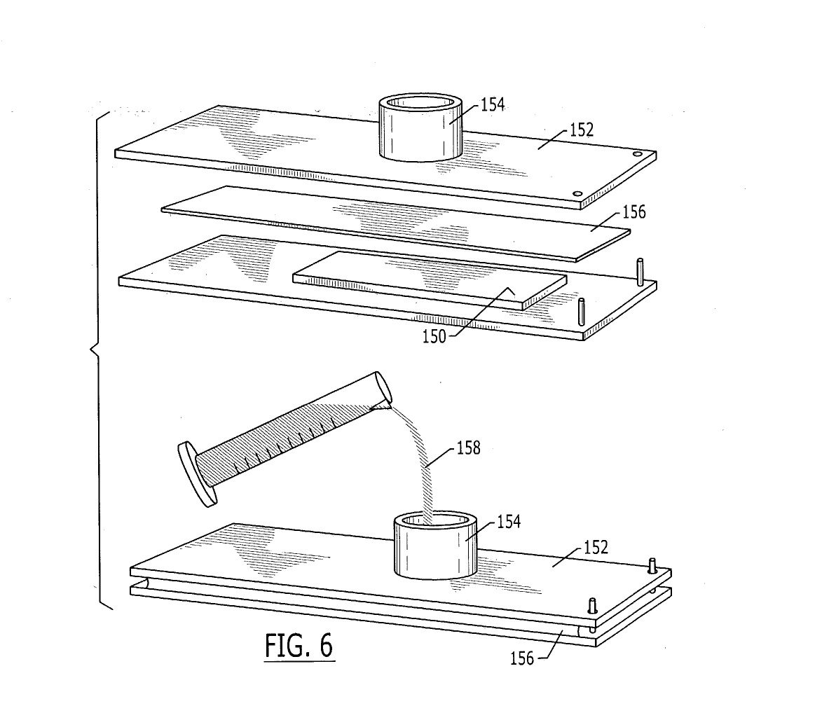

Table 1 provides both the recipes for and the properties exhibited by Examples 1 through 11 and Comparative Examples 1 through 8. The basis weight and density of each sample were determined using methods well known in the art. The acquisition, or intake, rates were determined using a standard intake rate test that measures the amount of time taken for a liquid to disappear from the surface of a sample. The apparatus used to determine the acquisition rate is schematically illustrated in FIG. 6. FIG. 6A provides an exploded view of the apparatus while FIG. 6B provides an illustration of the apparatus in use. As shown, the intake rate apparatus generally includes a 3″ by 6″ elevated anvil 150 and a top platen 152. The top platen 152, weighing 880 g, has a 2 inch hole connected to a tube 154. The top platen 152 is designed to apply a 0.1 psi load to the sample 156. To perform the intake rate test, a 300 mm by 110 mm sample 156 is placed between the elevated anvil 150 and the top platen 152. An initial liquid insult 158, i.e. approximately 100 ml of a 0.9% NaCl solution, is then introduced into the tube 154 and the time for the solution to disappear into the sample 156 is measured. The sample 156 is allowed to sit in the apparatus for 5 minutes and the insult/measurement procedure is repeated. In total, the insult/measurement procedure is repeated three times.

TABLE 1

| | | | | | | | | | Insult | Insult |
|---|---|---|---|---|---|---|---|---|---|---|
| | | | | Basis | | | | | 2/1 | 3/1 |
| Sample | Recipe | PET | SAP | Weight | Density | Intake Rate, mL/s | | | Rate | Rate |
| ID | ID | Type | Type | gsm | g/cc | Insult 1 | Insult 2 | Insult 3 | Ratio | Ratio |
| Comp. | A | | SXM | 447 | 0.37 | 0.93 | 0.45 | 0.42 | 0.48 | 0.45 |
| Ex. 1 | | | 880 | | | | | | | |
| Ex. 1 | B | 15 df, | SXM | 436 | 0.29 | 1.14 | 0.91 | 0.85 | 0.80 | 0.75 |
| | | solid | 880 | | | | | | | |
| Comp. | C | | SXM | 416 | 0.33 | 0.81 | 0.44 | 0.45 | 0.55 | 0.56 |
| Ex. 2 | | | 880 | | | | | | | |
| Ex 2 | D | 15 df, | SXM | 411 | 0.26 | 1.32 | 1.02 | 0.92 | 0.78 | 0.70 |
| | | solid | 880 | | | | | | | |
| Comp. | E | | SXM | 245 | 0.29 | 0.62 | 0.45 | 0.46 | 0.73 | 0.75 |
| Ex. 3 | | | 880 | | | | | | | |
| Ex. 3 | F | 15 df, | SXM | 248 | 0.23 | 0.88 | 0.79 | 0.79 | 0.90 | 0.90 |
| | | solid | 880 | | | | | | | |
| Comp. | D | | SXM | 457 | 0.28 | 1.21 | 0.83 | 0.81 | 0.69 | 0.67 |
| Ex. 4 | | | 9200 | | | | | | | |
| Comp. | D | | SXM | 461 | 0.32 | 1.06 | 0.66 | 0.70 | 0.62 | 0.66 |
| Ex. 5 | | | 9200 | | | | | | | |
| Ex. 4 | G | 9 df, | SXM | 460 | 0.25 | 1.57 | 1.92 | 2.06 | 1.22 | 1.31 |
| | | hollow | 9200 | | | | | | | |
| Ex. 5 | G | 9 df, | SXM | 475 | 0.30 | 1.49 | 1.78 | 1.79 | 1.20 | 1.20 |
| | | hollow | 9200 | | | | | | | |
| Ex. 6 | G | 15 df, | SXM | 454 | 0.28 | 1.37 | 1.48 | 1.52 | 1.08 | 1.11 |
| | | hollow | 9200 | | | | | | | |
| Ex. 7 | G | 15 df, | SXM | 451 | 0.34 | 1.23 | 1.20 | 1.13 | 0.98 | 0.92 |
| | | hollow | 9200 | | | | | | | |
| Comp. | H | | SXM | 444 | 0.32 | 1.16 | 0.80 | 0.85 | 0.69 | 0.74 |
| Ex. 6 | | | 9200 | | | | | | | |
| Comp. | J | | SXM | 439 | 0.30 | 1.24 | 1.12 | 1.08 | 0.90 | 0.87 |
| Ex. 7 | | | 9200 | | | | | | | |
| Ex. 8 | I | 15 df, | SXM | 463 | 0.30 | 1.46 | 2.26 | 1.93 | 1.55 | 1.32 |
| | | solid | 9200 | | | | | | | |
| Ex. 9 | G | 15 df, | SXM | 476 | 0.28 | 1.87 | 2.29 | 2.11 | 1.23 | 1.13 |
| | | solid | 9200 | | | | | | | |
| Comp. | H | | SXM | 464 | 0.37 | 1.12 | 0.97 | 1.01 | 0.86 | 0.90 |
| Ex. 8 | | | 9200 | | | | | | | |

| | | | | | | |
|---|---|---|---|---|---|---|
| Duocore ™ System¹ | 500 | 2.48 | 0.99 | 0.79 | 0.40 | 0.32 |
| Huggies Ultra-trim ™, | 850 | 2.12 | 1.16 | 1.20 | 0.55 | 0.56 |
| Step 4² | | | | | | |

¹Commercially available absorbent core from Buckeye Technologies of Memphis, Tennessee, insult amount was 75 ml. Data taken from http://beta.cecnet.com/bkiabsorb/html/unicore8902.html.
²Commercially available from Kimberly Clark of Neenah, WI.

As indicated in Table 1, absorbent cores formed in accordance with the present invention exhibit beneficial intake characteristics, such as initial acquisition rates, in comparison to comparable absorbent cores formed without synthetic fiber.

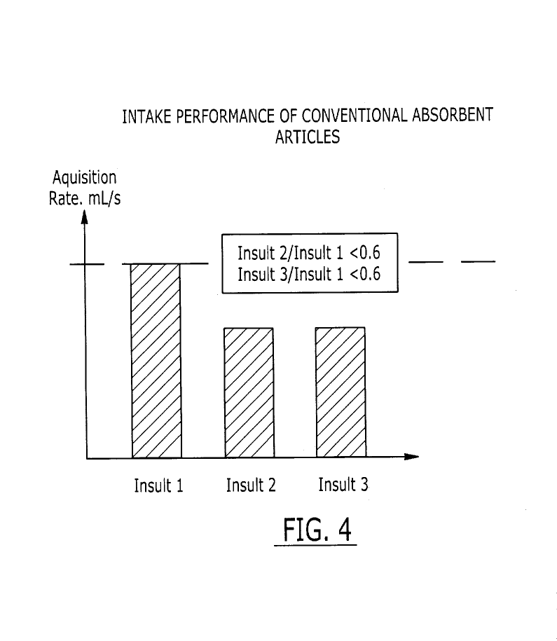

Further, the beneficial acquisition rates of the present invention do not deteriorate as dramatically after the initial insult as compared to comparable absorbent cores produced without synthetic fiber. In fact, in advantageous embodiments, the acquisition rate improves with successive insults, i.e. the ratio of the successive insults to the initial insult is greater than 1.0, which is altogether unexpected. In the case of absorbent cores made with conventional processes, such as pocket forming and thermal bonded airlaid, it has been found that during multiple insults the intake performance of absorbent cores starts decreasing dramatically, as indicated both by the performance of the HUGGIES ULTRATRIM™ and DUOCORE™ Samples provided in Table 1. As shown in Table 11, for conventional absorbent cores, the ratio of the acquisition rate for the 2^{nd} insult compared to the 1^{st} insult (i.e. the second insult ratio) and ratio of the acquisition rate for the 3^{rd} insult compared to the 1^{st} insult (i.e. the third insult ratio) is generally less than about 0.6. Consequently, upon multiple insults the ability of the absorbent core to rapidly acquire liquid starts diminishing, which in turn leads to increased pooling and leakage. The acquisition rate trend for conventional absorbent cores following multiple insults is also graphically represented in FIG. 4. The trend plotted in FIG. 4 can be expected in absorbent cores present in leading diapers such as HUGGIES ULTRA-TRIM™ or PAMPERS BABY DRY™ as well as air-laid absorbent cores such as those offered by Buckeye Technologies (under the brand name DUOCORE SYSTEM™).

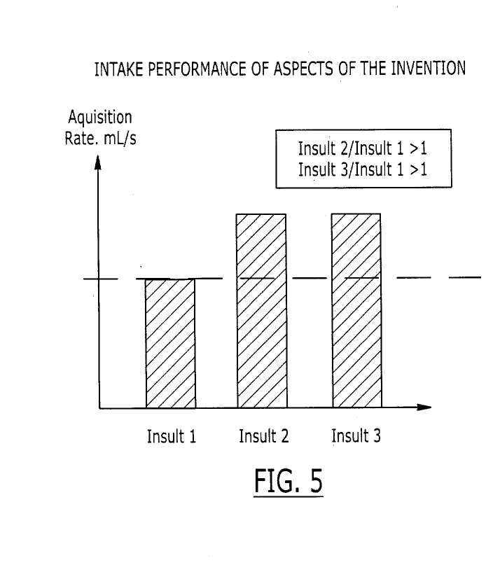

In contrast, the acquisition rates of the present absorbent cores do not diminish as rapidly. More particularly, in beneficial embodiments of the invention, the ratio of the acquisition rate for the 2^{nd} insult/1^{st} insult is greater than 0.9 and the ratio of the acquisition rate for the 3^{rd} insult/1^{st} insult is also greater than 0.9. Surprisingly, when Applicants included synthetic fibers in accordance with particularly advantageous embodiments of the present invention, the intake performance of the absorbent cores actually started improving after the first liquid insult, as indicated by several of the Examples in Table 11 and graphically illustrated in FIG. 5. More specifically, in particularly advantageous embodiments of the invention, the ratio of the acquisition rate for the 2^{nd} insult/1^{st} insult is greater than 1.0 and the ratio of the acquisition rate for the 3^{rd} insult/1^{st} insult is also greater than 1.0.

Examples 10 through 14 in accordance with present invention were produced using the layer compositions provided as Recipes K, L and M below. The specific recipe corresponding to each of Examples 10 through 14 is noted in Table 2. The samples were produced using 17 gsm tissue as the carrier layer, commercially available as designated grade 3008 from Cellu Tissue Corporation. The SAP used was SXM 9200, obtained from Stockhansen GmbH, Krefeld, Germany. The TAB nonwoven was a 40 gsm Libeltex grade T-9 carded through-air bonded nonwoven available from Libeltex in Meulebeke, Belgium. The cellulose fiber was untreated pulp fiber identified as RAYFLOC-J-LD pulp fiber, commercially available from Rayonier Inc. of Jesup, Ga.

The samples were made in accordance with the process shown in FIG. 3, except that a nonwoven sheet was introduced either between or prior to the forming heads, as indicated noted below. In addition to the nonwoven sheet, each of the absorbent core samples included airlaid material deposited by one or more forming heads, as noted within Recipes K, L and M. The configurations for the various recipes are described below:

| % in Each Forming Head | | | |
|---|---|---|---|
| | Nonwoven | | % of total |
| SAP | Type | Pulp | basis weight |

RECIPE K

| | | | | |
|---|---|---|---|---|
| Tissue | | | | 3% |
| Nonwoven | | TAB | | 8% |
| FH 1 | 63% | | 37% | 16% |
| FH 2 | 63% | | 37% | 16% |
| FH 3 | 63% | | 37% | 16% |
| FH 4 | 63% | | 37% | 16% |
| FH 5 | 63% | | 37% | 17% |
| FH 6 | | | 100% | 8% |

RECIPE L

| | | | | |
|---|---|---|---|---|
| Tissue | | | | 3% |
| FH 1 | 63% | | 37% | 16% |
| FH 2 | 63% | | 37% | 16% |
| FH 3 | 63% | | 37% | 16% |
| Nonwoven | | TAB | | 8% |
| FH 4 | 63% | | 37% | 16% |
| FH 5 | 63% | | 37% | 17% |
| FH 6 | | | 100% | 8% |

RECIPE M

| | | | | |
|---|---|---|---|---|
| Tissue | | | | 3% |
| FH 1 | 63% | | 37% | 16% |
| FH 2 | 63% | | 37% | 16% |
| FH 3 | 63% | | 37% | 16% |
| FH 4 | 63% | | 37% | 16% |
| FH 5 | 63% | | 37% | 17% |
| Nonwoven | | TAB | | 8% |
| FH 6 | | | 100% | 8% |

Table 2 provides the composition of and properties exhibited by Examples 10 through 14. The basis weight and density of each sample were again determined using methods well known in the art. The acquisition, or intake, rates were determined using the standard intake rate test described above.

TABLE 2

| | | | | | | | Insult | Insult |
|---|---|---|---|---|---|---|---|---|
| | | Basis | | Intake Rate, mL/s | | | 2/1 | 3/1 |
| Sample | Recipe | Weight | Density | Insult | Insult | Insult | Rate | Rate |
| ID | ID | gsm | g/cc | 1 | 2 | 3 | Ratio | Ratio |
| Ex. 10 | K | 469 | .36 | 1.26 | 1.24 | 1.03 | .98 | .82 |
| Ex. 11 | L | 470 | .29 | 1.52 | 1.73 | 1.30 | 1.14 | .86 |
| Ex. 12 | M | 466 | .29 | 1.28 | 1.11 | .96 | .87 | .75 |
| Ex. 13 | K | 480 | .34 | 1.27 | 1.26 | 1.11 | 1.00 | .87 |
| Ex. 14 | L | 467 | .27 | 1.63 | 2.10 | 1.67 | 1.29 | 1.02 |

As shown in Table 2, aspects of the invention incorporating pre-formed nonwoven sheet exhibited acquisition rate properties comparable to Examples 1 through 9. More particularly, all of the second and a majority of the third intake rates are at least 80% as fast as the first intake rate, as shown in Table 15. Surprisingly, samples in which the synthetic fiber was placed only in an intermediate layer provided beneficial acquisition rate properties as well.

Examples 15 through 17 in accordance with present invention were produced using the layer compositions provided as Recipes Q, R and U below. The specific recipe corresponding to each of Examples 15 through 17 is noted in Table 3. Comparative Example 9 was produced using the layer composition provided as Recipe W below. The samples were produced using 17 gsm tissue as the carrier layer, commercially available as designated grade 3008 from Cellu Tissue Corporation. This carrier tissue was placed on both the top and bottom of the web. The SAP used was ASAP 2260, obtained from BASF in Portsmouth, Va. The TAB nonwoven was a 40 gsm Libeltex grade T-9 carded through-air bonded nonwoven available from Libeltex in Meulebeke, Belgium. Pulp A was untreated cellulose pulp fiber, commercially available as RAYFLOC-J-LD pulp fiber from Rayonier Inc. of Jesup, Ga. Pulp B was cold caustic treated cellulosic fiber commercially available as POROSANIER-BAT from Rayonier Inc. of Jesup, Ga.

RECIPE Q

| | % in Each Forming Head | | | | % of total |
|---|---|---|---|---|---|
| | | Nonwoven | | | basis |
| | SAP | Type | Pulp A | Pulp B | weight |
| Tissue | | | | | 7% |
| FH 1 | 61% | | 39% | | 13% |
| FH 2 | 61% | | 22% | 17% | 13% |
| FH 3 | 61% | | 39% | | 13% |
| FH 4 | 61% | | 22% | 17% | 13% |
| FH 5 | 61% | | 39% | | 13% |
| Nonwoven | | TAB | | | 16% |
| FH 6 | 0% | | 100% | | 5% |
| Tissue | | | | | 7% |

RECIPE R

| | % in Each Forming Head | | | | % of total |
|---|---|---|---|---|---|
| | | Nonwoven | | | basis |
| | SAP | Type | Pulp A | Pulp B | weight |
| Tissue | | | | | 7% |
| FH 1 | 61% | | 39% | | 13% |
| FH 2 | 61% | | 22% | 17% | 13% |
| FH 3 | 61% | | 39% | | 13% |
| Nonwoven | | TAB | | | 16% |
| FH 4 | 61% | | 22% | 17% | 13% |
| FH 5 | 61% | | 39% | | 13% |
| FH 6 | 0% | | 100% | | 5% |
| Tissue | | | | | 7% |

RECIPE U

| | | | | | % of total |
|---|---|---|---|---|---|
| | % in Each Forming Head | | | | basis |
| | SAP | Nonwoven | Pulp A | Pulp B | weight |
| Tissue | | | | | 7% |
| FH 1 | 56% | | 44% | | 14% |
| FH 2 | 56% | | 24% | 20% | 14% |
| FH 3 | 56% | | 44% | | 14% |
| FH 4 | 56% | | 24% | 20% | 14% |
| FH 5 | 56% | | 44% | | 14% |
| Nonwoven | | TAB | | | 16% |
| FH 6 | | | 100% | | 7% |

RECIPE W

| | | | | | % of total |
|---|---|---|---|---|---|
| | % in Each Forming Head | | | | basis |
| | SAP | PET | Pulp A | Pulp B | weight |
| Tissue | | | | | 7% |
| FH 1 | 51% | | 49% | | 16% |
| FH 2 | 51% | | 27% | 22% | 15% |
| FH 3 | 51% | | 49% | | 16% |
| FH 4 | 51% | | 27% | 22% | 15% |
| FH 5 | 51% | | 49% | | 16% |
| FH 6 | | | 100% | | 8% |
| Tissue | | | | | 7% |

The composition and properties exhibited by Examples 15 through 17 are provided in Table 3. The acquisition rates for each of the samples were determined generally using the method described above. However, since Examples 15 through 17 and Comparative Example 9 have a relatively low basis weight, the acquisition rate test procedure was modified to use 55 g insults rather than the standard 100 g insults of the previous examples. The basis weights and densities were determined for the samples by methods well known in the art.

TABLE 3

| | | Basis | | | | | Insult 2/1 | Insult 3/1 |
|---|---|---|---|---|---|---|---|---|
| Sample | Recipe | Weight | Density | 55 ml Intake Rate, mL/s | | | Rate | Rate |
| ID | ID | gsm | g/cc | Insult 1 | Insult 2 | Insult 3 | Ratio | Ratio |
| Comp. | W | 243 | .27 | .72 | .45 | .38 | .63 | .53 |
| Ex. 9 | | | | | | | | |
| Ex. 15 | Q | 248 | .20 | .90 | .89 | .78 | .98 | .86 |
| Ex. 16 | R | 244 | .18 | 1.19 | 1.33 | 1.22 | 1.11 | 1.02 |
| Ex. 17 | U | 278 | .22 | 1.22 | 1.28 | 1.15 | 1.05 | .94 |

As shown in Table 3, all of the second intake rates and a majority of the third intake rates are at least 80% as fast as the intake rate on the first insult for Examples 15 through, 17. Further, the majority of Examples 15 through 17 exhibit overall improved acquisition rates (i.e. first and subsequent acquisition rates) over the control sample, Comparative Example 9. Again, surprising beneficial acquisition rate properties are provided by samples having synthetic fiber in the intermediate layers alone.

Examples 18 and 19 in accordance with present invention were produced using the layer compositions provided as Recipes T and V below. The specific recipe corresponding to a given example is noted in Table 4. The sample was produced using 17 gsm tissue as the carrier layer, commercially available as designated grade 3008 from Cellu Tissue Corporation. The SAP used in Examples 18 and 19 was ASAP 2260, obtained from BASF in Portsmouth, Va. The samples contained untreated cellulose pulp fiber, Pulp A, commercially available as RAYFLOC-J-LD pulp fiber from Rayonier Inc. of Jesup, Ga. The samples further contained cold caustic treated cellulosic fiber, Pulp B, commercially available as POROSANIER-BAT fiber from Rayonier Inc. of Jesup, Ga. The PET fibers were 15-denier type 224 in a 0.25 in. length from KOSA of Charlotte, N.C.

| | | | | % of total |
|---|---|---|---|---|
| % in Each Forming Head | | | | basis |
| SAP | PET | Pulp A | Pulp B | weight |

RECIPE T

| | | | | | |
|---|---|---|---|---|---|
| Tissue | | | | | 7% |
| FH 1 | 61% | | 39% | | 13% |
| FH 2 | 61% | | 22% | 17% | 13% |
| FH 3 | 61% | | 39% | | 13% |
| FH 4 | 61% | | 22% | 17% | 13% |
| FH 5 | 38% | 38% | 24% | | 21% |
| FH 6 | | 61% | 39% | | 13% |
| Tissue | | | | | 7% |

RECIPE V

| | | | | | |
|---|---|---|---|---|---|
| Tissue | | | | | 7% |
| FH 1 | 60% | | 40% | | 13% |
| FH 2 | 60% | | 19% | 21% | 13% |
| FH 3 | 60% | | 40% | | 13% |
| FH 4 | 60% | | 19% | 21% | 13% |
| FH 5 | 33% | 33% | 33% | | 24% |
| FH 6 | 0% | 50% | 50% | | 17% |

The acquisition rates for Examples 18 and 19 were also measured according to the method described above, again using 55 ml insults due to the lighter material basis weight. The results for Examples 18 and 19 are provided in Table 4.

TABLE 4

| | | | Basis | | | | | Insult 2/1 | Insult 3/1 |
|---|---|---|---|---|---|---|---|---|---|
| Sample | Recipe | PET | Weight | Density | 55 ml Intake Rate, mL/s | | | Rate | Rate |
| ID | ID | Type | Gsm | g/cc | Insult 1 | Insult 2 | Insult 3 | Ratio | Ratio |
| Ex. 18 | T | 15 df | 249 | .25 | .70 | .58 | .55 | .83 | .80 |
| | | Solid | | | | | | | |
| Ex. 19 | V | 15 df | 252 | .29 | .82 | .73 | .68 | .89 | .83 |
| | | Solid | | | | | | | |

Similar to the results from the previous examples, the second or third intake ratios for Examples 18 and 19 are at least 0.80. Again, Examples 18 and 19 indicate improved intake performance over Comparative Example 9 and highlight the beneficial aspects of the invention in which synthetic fiber is included within layers other than the innermost layer.

Many modifications and other embodiments of the inventions set forth herein will come to mind to one skilled in the art to which these inventions pertain having the benefit of the teachings presented in the foregoing descriptions and the associated drawings. Therefore, it is to be understood that the inventions are not to be limited to the specific embodiments disclosed and that modifications and other embodiments are intended to be included within the scope of the appended claims. Although specific terms are employed herein, they are used in a generic and descriptive sense only and not for purposes of limitation. For example, the term “or” is not used to indicate the associated elements or terms are mutually exclusive alternatives, rather the term “or” is used in a broader sense to mean that either or both elements or terms may be present.

That which is claimed:

1. An absorbent core comprising:
an innermost layer positioned towards the wearer;
at least one intermediate layer contiguous with said innermost layer and positioned away from the wearer, at least one of said intermediate layers comprising a mixture of cellulosic fiber and superabsorbent particles; and
an outermost layer contiguous with said intermediate layer and positioned furtherest from the wearer, said outermost layer comprising cellulosic fibers,
wherein either said innermost layer or at least one of said intermediate layers includes synthetic fiber in an effective amount and said absorbent core exhibits a second or third insult ratio of greater than 0.90.

2. An absorbent core according to claim 1, wherein said absorbent core exhibits a second or third insult ratio of greater than about 1.0.

3. An absorbent core according to claim 1, wherein said absorbent core has a density of greater than about 0.18 g/cm³.

4. An absorbent core according to claim 1, wherein said absorbent core has a density ranging from about 0.20 to 0.50 g/cm³.

5. An absorbent core comprising:
an innermost layer positioned towards the wearer, said innermost layer including synthetic fiber in an amount effective to improve the second or third insult ratio of said absorbent core in comparison to a comparable absorbent core without synthetic fiber;
at least one intermediate layer contiguous with said innermost layer and positioned away from the wearer, at least one of said intermediate layers comprising a mixture of cellulosic fiber and superabsorbent particles; and
an outermost layer contiguous with said intermediate layer and positioned furtherest from the wearer, said outermost layer comprising cellulosic fibers.

6. An absorbent core according to claim 5, wherein said synthetic fiber comprises at least one polymer selected from the group consisting of polyakylene terephthalate, polyolefin, acrylic, polyamide, rayon and acetate.

7. An absorbent core according to claim 5, wherein said synthetic fiber is polyethylene terephthalate.

8. An absorbent core according to claim 5, wherein said synthetic fiber is present in said innermost layer in an amount ranging from about 20 to 100 weight percent, bol.

9. An absorbent core according to claim 5, wherein said synthetic fiber has a denier ranging from about 3 to 25 dpf.

10. An absorbent core according to claim 5, wherein said synthetic fiber has been hydrophilicly modified.

11. An absorbent core according to claim 5, wherein said synthetic fiber is a multicomponent fiber.

12. An absorbent core according to claim 5, wherein said innermost layer further comprises cellulosic fiber.

13. An absorbent core according to claim 5, wherein said innermost layer further comprises super absorbent particles.

14. An absorbent core according to claim 5, wherein said innermost layer forms from about 3 to 20 weight percent of said absorbent core.

15. An absorbent core according to claim 5, wherein said cellulosic fiber is derived from wood pulp, cotton, flax or peat moss.

16. An absorbent core according to claim 5, wherein said cellulosic fiber is present in said intermediate layer in an amount ranging from about 20 to 100 weight percent, bol.

17. An absorbent core according to claim 5, wherein said cellulosic fibers comprise a mixture of untreated and alkaline treated cellulosic fibers.

18. An absorbent core according to claim 5, wherein said alkaline treated cellulosic fibers are present in said intermediate layer in an amount ranging from about 15 to 25 weight percent, bol.

19. An absorbent core according to claim 5, wherein said superabsorbent particles comprise a salt of a crosslinked polyacrylic acid.

20. An absorbent core according to claim 5, wherein said superabsorbent particles are present in said intermediate layer in an amount ranging from about 5 to 67 weight percent, bol.

21. An absorbent core according to claim 5, wherein said intermediate layer forms from about 20 to 90 weight percent of said absorbent core.

22. An absorbent core according to claim 5, wherein said outermost layer forms from about 2 to 15 weight percent of the absorbent core.

23. An absorbent core according to claim 5, wherein said absorbent core comprises a plurality of intermediate layers.

24. An absorbent core according to claim 23, wherein said plurality of intermediate layers comprises a first intermediate layer contiguous with said innermost layer, a second intermediate layer contiguous with said first intermediate layer, and a third intermediate layer contiguous with said second intermediate layer.

25. An absorbent core according to claim 24, wherein said superabsorbent particles are included in greater amounts in second and third intermediate layers than in said first intermediate layer.

26. An absorbent core according to claim 24, wherein said superabsorbent particles are not present in said first intermediate layer.

27. An absorbent core according to claim 24, wherein said second and third intermediate layers exhibit a higher basis weight than said first intermediate layer.

28. An absorbent core according to claim 24, further comprising a fourth intermediate layer contiguous with said third intermediate layer, said fourth intermediate layer contiguous with said outermost layer.

29. An absorbent core according to claim 28, wherein said innermost layer forms from about 5 to 33 weight percent of the absorbent core.

30. An absorbent core according to claim 28, wherein said innermost layer comprises synthetic fiber in an amount ranging from about 20 to 100 weight percent, bol.

31. An absorbent core according to claim 28, wherein said first intermediate layer forms up to about 50 weight percent of the absorbent core.

32. An absorbent core according to claim 28, wherein said first intermediate layer comprises cellulosic fiber in an amount ranging from about 15 to 100 weight percent, bol.

33. An absorbent core according to claim 28, wherein said first intermediate layer comprises superabsorbent particles in an amount of up to 85 weight percent, bol.

34. An absorbent core according to claim 28, wherein said first intermediate layer comprises synthetic fiber in an amount of up to 50 weight percent, bol.

35. An absorbent core according to claim 28, wherein said second intermediate layer forms up to about 33 weight percent of the absorbent core.

36. An absorbent core according to claim 28, wherein said second intermediate layer comprises cellulosic fiber in an amount ranging up to about 60 weight percent, bol.

37. An absorbent core according to claim 28, wherein said second intermediate layer comprises superabsorbent particles in an amount ranging up to about to 60 weight percent, bol.

38. An absorbent core according to claim 28, wherein said second intermediate layer comprises synthetic fiber in an amount ranging from about 20 to 100 weight percent, bol.

39. An absorbent core according to claim 28, wherein said third and fourth intermediate layers independently form from about 12 to 70 weight percent of the absorbent core.

40. An absorbent core according to claim 28, wherein said third and fourth intermediate layers comprise cellulosic fiber in an amount ranging independently from about 10 to 66 weight percent, bol.

41. An absorbent core according to claim 28, wherein said third and fourth intermediate layers comprise superabsorbent particles in an amount ranging independently from about 33 to 90 weight percent, bol.

42. An absorbent core according to claim 28, wherein said third and fourth intermediate layers comprise synthetic fiber in an amount ranging independently from about 5 to 100 weight percent, bol.

43. An absorbent core according to claim 28, wherein said absorbent core exhibits a second or third insult ratio of greater than about 1.0.

44. An absorbent core according to claim 5, further comprising a carrier layer.

45. An absorbent core according to claim 5, wherein said absorbent core has a basis weight of about 450 gsm.

46. An absorbent core according to claim 5, wherein said absorbent core has a basis weight of about 250 gsm.

47. An absorbent core according to claim 5, wherein said carrier layer comprises tissue.

48. An absorbent core according to claim 5, wherein said synthetic fiber is in the form of a through-air-bonded, point-bonded, spun-bonded or resin-bonded pre-formed nonwoven sheet.

49. An absorbent core according to claim 48, wherein said pre-formed nonwoven sheet comprises synthetic fiber exhibiting a denier ranging from about 3 to 25 dpf.

50. An absorbent core according to claim 48, wherein said pre-formed nonwoven sheet forms from about 4 to 32 weight percent of said absorbent core.

51. An absorbent core comprising:
an innermost layer comprising cellulosic fiber positioned towards the wearer;
at least one intermediate layer contiguous with said innermost layer and positioned away from the wearer, at least one of said intermediate layers including synthetic fiber in an amount effective to improve the second or third insult ratio of said absorbent core in comparison to a comparable absorbent core without synthetic fiber; and
an outermost layer comprising cellulosic fiber contiguous with said intermediate layer and positioned furtherest from the wearer.

52. An absorbent core according to claim 51, wherein said synthetic fiber comprises at least one polymer selected from the group consisting of polyakylene terephthalate, polyolefin, acrylic, polyamide, rayon and acetate.

53. An absorbent core according to claim 51, wherein said synthetic fiber is polyethylene terephthalate.

54. An absorbent core according to claim 51, wherein said synthetic fiber is present in an amount ranging from about 5 to 100 weight percent, bol.

55. An absorbent core according to claim 51, wherein said synthetic fiber has a denier ranging from about 3 to 25 dpf.

56. An absorbent core according to claim 51, wherein said intermediate layer including synthetic fiber is a through-air-bonded, point-bonded, spun-bonded, needle-punched or resin-bonded pre-formed nonwoven sheet.

57. An absorbent core according to claim 56, wherein said pre-formed nonwoven sheet comprises synthetic fiber exhibiting a denier ranging from about 3 to 25 dpf.

58. An absorbent core according to claim 56, wherein said pre-formed nonwoven sheet forms from about 4 to 32 weight percent of said absorbent core.

59. A method for producing an absorbent core comprising:
(a) forming an innermost layer by directing a plurality of discrete length fibers onto a collection surface;
(b) forming at least one intermediate layer by directing a plurality of discrete length fibers onto the innermost layer;
(c) forming an outermost layer by directing a plurality of discrete length fibers onto the intermediate layer;
(d) compacting the innermost, intermediate and outermost layers to form a condensed web; and
(e) calendaring the condensed web, thereby forming a unitary structure, wherein at least one of said innermost and intermediate layers includes synthetic fiber in an amount effective to improve the second or third insult ratio of the absorbent core in comparison to a comparable absorbent core without synthetic fiber.

60. A method according to claim 59, wherein said step of forming an innermost layer further comprises directing a plurality of discrete length synthetic fibers onto a collection surface.

61. A method according to claim 59, further comprising inserting a carrier layer between the innermost layer and the collection surface.

62. A method according to claim 59, further comprising inserting a pre-formed nonwoven web between the innermost layer and the collection surface.

63. A method according to claim 59, further comprising inserting a pre-formed nonwoven web as an intermediate layer between the innermost and outermost layers.

64. An absorbent article comprising an absorbent core which includes:
an innermost layer positioned towards the wearer;
at least one intermediate layer contiguous with said innermost layer and positioned away from the wearer, at least one of said intermediate layers comprising a mixture of cellulosic fiber and superabsorbent particles; and
an outermost layer contiguous with said intermediate layer and positioned furtherest from the wearer, said outermost layer comprising cellulosic fibers,
wherein either said innermost layer or at least one of said intermediate layers includes synthetic fiber in an effective amount and said absorbent core exhibits a second or third insult ratio of greater than 0.90.

65. An absorbent article according to claim 64, wherein the absorbent article is selected from the group consisting of a diaper, a feminine hygiene product and an incontinence pad.
